(19) United States
(12) Patent Application Publication
(10) Pub. No.: US 2019/0339266 A1
(43) Pub. Date: Nov. 7, 2019

(54) MEASUREMENTS OF PROTEIN-PROTEIN INTERACTIONS AT SINGLE MOLECULE LEVEL

(71) Applicant: Bio-Rad Laboratories, Inc., Hercules, CA (US)

(72) Inventor: Ning LIU, Walnut Creek, CA (US)

(21) Appl. No.: 16/475,247

(22) PCT Filed: Dec. 19, 2017
(86) PCT No.: PCT/US2017/067327

§ 371 Date: Jul. 1, 2019

Related U.S. Application Data
(60) Provisional application No. 62/452,231, filed on Jan. 30, 2017.

(51) Int. Cl. G01N 33/557; G01N 33/52; G01N 33/68; G01N 21/64; G06T 7/00; G06T 7/13

(52) U.S. Cl. CPC G01N 33/557; G01N 33/52; G06T 7/13; G01N 21/648; G06T 7/0014; G01N 33/68

(57) ABSTRACT

Methods for characterizing the binding characteristic of at least one protein molecule to a detectably labeled probe are provided.

### CROSS-REFERENCE TO RELATED PATENT APPLICATIONS

The present patent application claims benefit of priority to U.S. Provisional Patent Application No. 62/452,231, filed Jan. 30, 2017, which is incorporated by reference for all purposes.

### BACKGROUND

Immunoassays are widely adopted methods to determine quantity and/or presence of molecules in biological and other samples. These methods rely on highly selective interactions between antibodies and antigens. The sensitivity of immunoassay is a key performance indicator of good assays which is affected by both constant and variable sources of noise of the assay platform. The constant sources of noise, arising from detection elements, signal processing, molecular shot noise, thermal noise, and Johnson noise will be present through any measurement at a defined intensity for a particular system (Woolley et al., 2015, Anal Bioanal Chem 407:8605-8615). However, with improving detection technologies capable of routine single molecule detection, the instrumentation to detect antibody-antigen binding is no longer a fundamental factor that defines the limit of detection for immunoassays. Variable noise arises predominantly from non-specific binding of antibodies, which occurs at each step in immunocomplex formation with different effects. Although binding of the non-target species is less probable than the specific binding of an analyte, in the event of a low concentration, non-specific binding is a significant contributor to the overall signal (Jackson and Ekins, 1986, J Immunological methods, 87:13-20; Hassibi et al, 2007, J Applied Physics, 102; Rissin et al., 2010 Nature Biotechnology 28:595-599; Schmidt et al, 2011 J Proteome Research 10:1316-1322; Chang et al., 2012, J immunological methods 378:102-115). Immunoassays are typically limited to quantitation in the nano- to picomolar range by non-specific binding effects.

Most of the commercially available immunoassays rely on the use of chemically linked fluorophores to antibodies. Fluorescent intensities of all antibodies bound to the assay platform, whether through specific or non-specific binding, are averaged and summed to generate an estimate of antigen concentration. The most effective approaches to reduce non-specific binding noise are to use highly specific and high affinity antibodies, to use a blocker, and to wash off the non-specifically bound antibodies as much as possible.

The prior art is limited in that it does not enable the use of low affinity or fast-off rate probes to study protein-protein interactions at single molecule level. There is a need to complement or supplement existing tools, such as Surface Plasmon Resonance (SPR), 2-D gel electrophoresis, and mass spectrometry (MS), due to the need for enhanced sensitivity and specificity.

### BRIEF SUMMARY OF THE INVENTION

The present invention provide methods for characterizing biomolecules such as proteins, as well as detecting binding characteristics between binding pair members at the single molecule level. Among other things, these methods provide a solution to distinguish specific binding of a probe to its antigen from non-specific binding of the probe to any non-target antigen molecules.

In one embodiment, a method for characterizing the binding characteristic of at least one protein molecule to a detectably labeled probe, the method comprising: contacting the protein molecules with a detectably labeled probe which exhibits fast-off rate binding characteristics with respect to the protein, to generate a transient binding interaction between the protein molecule and the probe; detecting the transient interactions by single molecule detection of the labeled probe at plurality of locations, wherein the transient interactions have an observed residence time of about 10 minutes to about 1 nanosecond; and correlating the interaction frequencies of multiple transient interactions at the plurality of locations to determine the binding characteristic of the protein molecule to the probe.

In one embodiment, the protein is immobilized on a surface.

In one embodiment, the immobilized surface is glass, quartz or plastic.

In one embodiment, the protein is immobilized on the surface using a hydrophilic self-assembled monolayer, a hydrophilic polymer brush, a zwitterionic polymer brush or a nitrile coating.

In one embodiment, the surface is coated with streptavidin and the protein is immobilized to the surface using biotinylated protein.

In one embodiment, the protein is immobilized at a surface density of about 2 molecules to about 1×10⁶ molecules per 100 μm².

In one embodiment, the protein is immobilized at a surface density of about 2×10² molecules to about 8×10⁵ molecules per 100 μm².

In one embodiment, the protein is immobilized at a surface density of about 2×10³ molecules to about 6×10⁴ molecules per 100 μm².

In one embodiment, correlating the interaction frequencies of multiple transient interactions at the plurality of locations allows separation of protein-specific signal from nonspecific noise.

In one embodiment, the ratio of protein-specific signal interactions to nonspecific noise interactions is more than or about 2.

In one embodiment, the ratio of protein-specific signal interactions to nonspecific noise interactions is more than or about 3.

In one embodiment, the ratio of protein-specific signal interactions to nonspecific noise interactions is more than or about 4.

In one embodiment, correlating the interaction frequencies of multiple transient interactions at the plurality of locations measures total number of on- and off-events at plurality of locations.

In one embodiment, more than 1000 transient interactions are recorded per viewing area.

In one embodiment, more than 2000 transient interactions are recorded per viewing area.

In one embodiment, more than 4000 transient interactions are recorded per viewing area.

In one embodiment, the binding characteristic comprises a statistical metric calculated from the multiple transient interactions between the probe and the protein molecules at the plurality of locations.

In one embodiment, the statistical metric is calculated from Poisson statistics, hidden Markov modeling or an edge detection algorithm.

In one embodiment, the statistical metric comprises one or more of:
a. a mean of a distribution of residence times measured for the repeated binding of the probes with the protein molecules at plurality of locations;
b. a median of a distribution of residence times measured for the repeated binding of the probes with the protein molecules at plurality of locations;
c. a standard deviation of a distribution of residence times measured for the repeated binding of the probes with the protein molecules at plurality of locations;
d. a peak of a distribution of residence times measured for the repeated binding of the probes with the protein molecules at plurality of locations;
e. a shape of a distribution of residence times measured for the repeated binding of the probes with the protein molecules at plurality of locations;
f. a mean of a distribution of number of transient events measured for the repeated binding of the probes with the protein molecules at plurality of locations;
g. a median of a distribution of number of transient events measured for the repeated binding of the probes with the protein molecules at plurality of locations;
h. a standard deviation of a distribution of number of transient events measured for the repeated binding of the probes with the protein molecules at plurality of locations;
i. a peak of a distribution of number of transient events measured for the repeated binding of the probes with the protein molecules at plurality of locations; or
j. a shape of a distribution of number of transient events measured for the repeated binding of the probes with the protein molecules at plurality of locations.

### BRIEF DESCRIPTION OF THE DRAWINGS

FIG. 1(A) shows a thermodynamic profile of the IL-6 antibody interacting with a spot through non-specific binding. The total number of on- and off-events of the antibody at this spot is 6.

FIG. 1(B) shows a thermodynamic profile of the IL-6 antibody interacting with an IL-6 molecule on the surface. The total number of on- and off-events of the antibody at this spot is 24, significantly higher than the number for a non-specific spot.

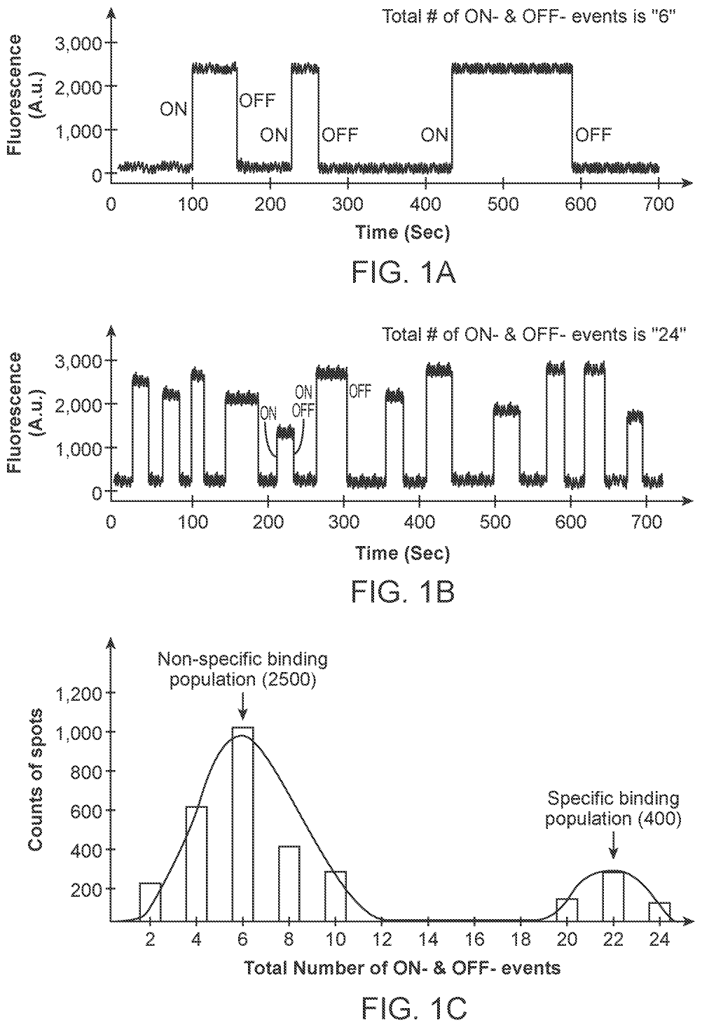

In FIG. 1(C), the IL-6 antibody interacted with a total of 2900 individual locations on the assay surface and generated 2900 individual thermodynamic profiles. Out of these, 2500 profiles (non-specific binding population) showed low probability of the IL-6 antibody interacting with them. There were a total of 2-10 on- and off-events observed for these spots. For the rest of the 400 spots (specific binding population), the IL-6 antibody actively seeked interaction with them which is demonstrated by a total of 20-24 on- and off-events. This results show that 400 target molecules are detected on the assay surface.

### DETAILED DESCRIPTION OF THE INVENTION I

### Concepts

Two terms frequently arise in discussions of recognition. Specificity measures the degree to which the immune system differentiates between different antigens. Cross-reactivity measures the extent to which different antigens appear similar to the immune system.

Specificity defines another dimension of immune recognition. Specificity is the degree to which an immune response discriminates between antigenic variants. A simple approach measures the relative binding affinities of purified antibodies or T cell receptors for different antigens.

Relatively low-affinity binding can often provide greater specificity when measured at intermediate stringency. This occurs because low-affinity receptors bind fewer kinds of antigens as conditions limit the assay's sensitivity for low-affinity binding. Thus, the relative specificity of different antibodies or T cells depends on both affinity and conditions of measurement.

The binding characteristics of an antigen to its antibody and their effects on off rate vs. total dissociation time are illustrated below. Thus, without wishing to be bound by theory, it is believed that fast off-rate antibodies (or other suitable binding partners) may be more suitable in these applications than the slow-off rate antibodies.

### Examples of K_{off}Rates

| | kₒₙ, M⁻¹ sec⁻¹ | k_{off}, sec⁻¹ | K_{D}, nM |
|---|---|---|---|
| a-TSH | 9.0 × 10⁴ | 1.1 × 10⁻³ | 12 |
| a-HGF | 9.5 × 10³ | 4.5 × 10⁻⁴ | 48 |
| a-EGF | 2.6 × 10⁴ | 1.3 × 10⁻³ | 51 |
| a-Leptin | 3.9 × 10³ | 1.3 × 10⁻⁴ | 33 |
| a-FADD | 2.1 × 10³ | 1.7 × 10⁻⁴ | 80 |

### Effect of K_{off} on Total Dissociation

| k_{off} | Tme to 90% Disscociation | |
|---|---|---|
| 0.1 | 23 | s |
| 1.E−02 | 3.8 | min. |
| 1.E−03 | 38 | min |
| 1.E−04 | 16 | days |
| 1.E−05 | 160 | days |

Herein is described a novel and more sensitive assay to minimize or eliminate the non-specific binding or noise utilizing single molecule detection technologies. The assay depends on the measurement of the probability of an antibody binding to all molecules, including its cognate antigens and other molecules, on the assay surface/platform. We observe/measure the thermodynamic interactions of the antibody/probe with each single molecule on the assay surface/platform for a certain period of time. This approach allows us to digitally count the number of specific antigens on the assay surface/platform, even in the presence of a much larger number of non-specific binding events, because the thermodynamic profile of a specific interaction is expected to be different from a non-specific one. This minimization or elimination of the non-specific noise leads to a much lower limit of quantification.

### Definitions

“Biomolecule” as used herein includes any type of biomolecule for which detection (including quantitative detection) may be desired, including but not limited to, peptides, proteins, nucleic acids, sugars, mono- and polysaccharides, lipids, lipoproteins, whole cells, and the like.

“Binding pair” as used herein includes a pair of molecules, one of which can be a probe and the other one can be a target molecule, which members of the pair of molecules can bind to one another with different affinities or not at all. Examples of suitable binding pairs include, but are not limited to, nucleic acid and nucleic acid; protein or peptide and nucleic acid; protein or peptide and protein or peptide; antigens and antibodies; receptors and ligands, haptens, or polysaccharides, complementary nucleic acids, pharmaceutical compounds, and the like.

The term “detect” or “detection” as used herein includes the determination of the existence, presence or fact of a target protein or signal in a limited portion of space, including but not limited to, a sample, a protein, a biomolecule, a binding event, a reaction mixture, a molecular complex and a substrate. A detection refers, relates to, or involves the measurement of quantity, amount or identity of the target protein or signal (also referred as quantitation), which includes but is not limited to, any analysis designed to determine the presence, absence, amounts or proportions of the target or signal. Detection also refers, relates to, or involves identification of a quality or kind of the target protein or signal in terms of relative abundance to another target or signal.

“Protein” or “polypeptide” includes a polymer of amino acid residues. These terms also apply to amino acid polymers in which one or more amino acid residues is an artificial chemical analogue of a corresponding naturally occurring amino acid, as well as naturally occurring amino acid polymers. An amino acid polymer in which one or more amino acid residues is an “unnatural” amino acid, not corresponding to any naturally occurring amino acid, is also encompassed by the use of the team “protein” and “polypeptide” herein.

The term “target” or “target molecule” or protein target as used herein includes an analyte of interest.

The term “analyte” includes a protein, substance, compound or component whose presence or absence in a sample has to be detected. Analytes include, but are not limited to, biomolecules and in particular proteins, or biomarkers.

The term “biomolecule” as used herein indicates a substance compound or component associated to a biological environment including, but not limited to, sugars, amino acids, peptides, proteins, oligonucleotides, polynucleotides, polypeptides, organic molecules, haptens, epitopes, biological cells, parts of biological cells, vitamins, hormones and the like.

The term “biomarker” indicates a biomolecule that is associated with a specific state of a biological environment including, but not limited to, a phase of cellular cycle, or health and disease state. The presence, absence, reduction, up regulation or down regulation of the biomarker is associated with and is indicative of a particular state.

The term “probe” as used herein includes a molecule which binds to another molecule (the target) in a binding pair, which probe molecule can be used to determine the presence or absence of the other molecule (i.e., target) The term “probe” is an agent including a binder and a unique label (e.g., a signaling moiety). In some embodiments, the binder and the signaling moiety of the probe are embodied in a single entity (e.g., a fluorescent molecule capable of binding a target). In certain instances, a probe can non-covalently bind to one or more protein targets in the biological sample. In certain instances, a probe can specifically bind to a target. Probes can be any member of a binding pair and include, for example, natural or modified peptides, proteins (e.g., antibodies, affibodies, or aptamers), nucleic acids (e.g., polynucleotides, DNA, or RNA); polysaccharides (e.g., lectins, sugars), lipids, enzymes, enzyme substrates or inhibitors, ligands, receptors, antigens, haptens, or synthetic nucleic acids such as DNA, RNA, small molecules, and the like.

The term “probe type” as used herein includes a descriptor to uniquely categorize a population of probe molecules having identical probe characteristics.

The term “target type” as used herein includes a descriptor to uniquely categorize a population of target molecules having identical target characteristics.

The term “panel of probes” as used herein includes a population of molecules selected from one or more probe types.

### EMBODIMENTS

Probes are allowed to contact a surface with immobilized proteins. In certain aspects, probes interact transiently with many different proteins immobilized on a surface, binding some proteins for a long time before unbinding, while binding other proteins for a shorter time period, and still other proteins not at all. The transient binding events at the surface are imaged by a fluorescence microscope, such as a total internal reflection fluorescence (TIRF) microscope. Time trace data of individual pixels is extracted by image analysis software, for example, revealing that probes reside for longer periods (on) at some spots than at other locations or revealing that probes bind at a different frequency at some spots than at other locations. In certain instances, a suitable probe can be selected depending on the sample of proteins to be analyzed and available for detection. For example, a target protein can include a receptor and the probe can include a ligand. Similarly, a target protein can include an antibody or antibody fragment and a probe can include an antigen.

It will be apparent to those of skill in the art that the following equation K_{d}=k_{off}/kₒₙ preferably characterizes transient binding. Any suitable metric of the transient binding of the probe, including K_{d}, k_{off} as well as others are suitable to generate a histogram. These include manipulations and any proxy of this equation K_{d}=k_{off}/kₒₙ. The observed residence times of the binding events of a probe(s) with a target may be on a time frame of about 1 nanosecond to about 10 minutes or more.

Binding frequency can be measured by the total number of on- and off-event observed at a specific spot in a time frame from about 1 nanosecond to about 10 min, 1 hour or even a day. Different patterns of transient binding of the probe to the immobilized biomolecules on the surface may be described by a combination of residence time and interaction frequency, and/or other parameters such as period of off-time, signal intensity etc. The residence time of the binding is different from how long an experimental set up may be operated which may include many binding events with differing residence time.

### Probes

In certain embodiments, a probe is an agent that is capable of binding a target protein. Typically, a probe comprises a signaling moiety such a fluorophore. Probes can be any member of a binding pair and include, for example, natural or modified peptides, proteins (e.g., antibodies, affibodies, nanobodies or aptamers), nucleic acids (e.g., polynucleotides, DNA, or RNA); polysaccharides (e.g., lectins, sugars), lipids, enzymes, enzyme substrates or inhibitors, ligands, receptors, antigens, haptens, or synthetic nucleic acids such as DNA, RNA, small molecules, and the like. The probes can be, for example, organic or inorganic molecules.

In addition, the probes can be formed by synthetic molecules. (Iterative In Situ Click Chemistry Creates Antibody-like Protein-Capture Agents, H. D. Agnew et al., Angew. Chem. Int. Ed. 2009, 48, 4944-4948.) (Accurate MALDI-TOF/TOF Sequencing of One-Bead-One-Compound Peptide Libraries with Application to the Identification of Multiligand Protein Affinity Agents Using in Situ Click Chemistry Screening, Su Seongi Lee et al., Anal. Chem., 2010, 82 (2), pp 672-679.)

In certain aspects, a probes is selected from random libraries of peptides, small ligands, small molecules and the like. In one instance, candidate probe agents can be, peptides, polypeptides, peptidomimetics, amino acids, amino acid analogs, saccharides, fatty acids, steroids, purines, pyrimidines, derivatives or structural analogues thereof, polynucleotides, and polynucleotide analogs. In certain instances, the panel of probes can be homogeneous or heterogeneous. That is, the panel can comprise the same members (homogeneous panel of one probe type) or different probe members (heterogeneous panel selected from more than one probe type). In other words, the panel can be all of one molecular type, or different molecular types.

In certain other instances, the probes are derived from a random library of peptides such as those that are commercially available, or generated using combinatorial techniques well known to those of skill in the art. Preferably, the individual probes in the panel of probes have a spectrum of binding affinities, so within the panel there are weak binding affinities (i.e. a low binding profile for a specific target bound to the surface) and strong binding probes (probes that bind tightly to the target), as well as intermediate binding affinities. Thus, the probes bind with enough diversity to generate a “signature.”

In most instances, the individual probes will have a unique label associated therewith. In certain preferred instances, a signaling moiety such as a fluorophore is covalently attached. Suitable fluorophores include, but are not limited to, 4-acetamido-4′-isothiocyanatostilbene-2,2′ disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives: coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; terbium chelate derivatives; Cy 3; Cy 5; Cy 5.5; Cy 7; IRD 700; IRD 800; La Jolla Blue; phthalo cyanine; naphthalo cyanine ATT0647; and IRDye 680LT.

In other instances, other fluorophores are suitable for use. These include, for example, green fluorophores (for example Cy3, FITC, and Oregon Green), which can be characterized by their emission at wavelengths generally in the range of 515-540 nanometers, and red fluorophores (for example Texas Red, Cy5, and tetramethylrhodamine), which can be characterized by their emission at wavelengths generally in the range of 590-690 nanometers. In one preferred aspect, a highly-photostable silicon-phthalocyanine dye IRDye, 700DX, which is notable not only for its photostability, but also for its high water-solubility and protein-phobic (non-stick) properties, can also be imaged with high sensitivity, and superior photostability.

In one aspect, probe selection criteria can be used to select the proper probe. These criteria include, for example, whether a candidate probe interacts too strongly with the bare assay surface. Further criteria is whether a candidate probe interacts with only a few proteins, or whether a candidate probe interacts with proteins on time scales too short for sampling by the optical system, or too long for recording multiple events in a predetermined period. In certain aspects, the probes are structurally diverse. Examples of diversity in peptide interactions include phage display where the selected peptides often have affinities too low for practical purposes (Choi, S. J. et al., Mol Cells, 7(5), 575-81 (1997)), and in work screening biotin peptide mimics by methodically varying a peptide sequence at two amino acids (Schmidt, T. G. et al., J Mol Biol, 255(5), 753-66 (1996)). More recently, use of lanthanide (Eu, Tb, Dy and Sm), chelates has been described for use in immunoassays to generate luminescence that exhibit longer emission output and less sensitivity to bleaching (Hagan and Zuchner Anal Bioanal Chem (2011) 400:2847). Such lanthanide-labeled probes are contemplated in present assays. Similarly, quantum dot and P-Dots also may be used.

In one embodiment, proteins from a sample are randomly immobilized on a surface at resolvable surface densities of up to 800,000 protein molecules per 100×100 μm field. A fluorescent probes is applied, and the surface is imaged using a total internal reflection fluorescence (TIRF) microscope to record movies of individual probes binding transiently with individual immobilized proteins. For each probe, a characteristic distribution of transient times is obtained from the traces of transient interactions recorded at each protein location for an assay time of about 10 min to about one day. The transient time distribution spectrum embodies the integrated affinity of the probe for the target protein. The total number of on- and off-events is also used as a characteristic of the probe binding to a specific protein location. Low probe concentrations minimize fluorescence background from unbound probes while still promoting reasonable binding rates at the surface. Low probe affinities imply rapid unbinding. The system can thus record multiple on-off (transient) events in order to estimate the distribution of transient times in reasonable assay time.

In certain instances the probe is a peptide such as an RGD peptide (arginine-glycine-aspartic acid), which is specific for an integrin. In other instances, the probe is a small molecule, an aptamer or an antibody.

In certain embodiments, probes are present at concentrations at about 1M to about 0.001 nM or even 1 pM, preferably about 1 mM to about 100 mM, more preferably at about 1 μM to about 100 μM and most preferably, 0.1 nM to about 5 nM such as 0.1, 0.2, 0.3, 0.4, 0.5, 0.6, 0.7, 0.8. 0.9, 1, 2, 3, 4, or 5 nM.

In certain embodiments, the technology provides a detection complex for detecting a protein or a portion of a protein. The detectably labeled (e.g. fluorescent) query probe that binds to the target protein has a kinetic rate constant K_{off} that is greater than 0.1 min⁻¹ and/or a kinetic rate constant Kₒₙ that is greater than 0.1 min⁻¹. For example, in some embodiments, the kinetic rate constant Kₒₙ describing the association of the query probe with the query region of the target protein to for a complex and/or the kinetic rate constant K_{off} describing the dissociation of the complex is/are great that 0.1 min⁻¹, e.g., greater than 1 min⁻¹ (e.g., greater than approximately 0.002 s⁻¹. Great than approximately 0.02 s⁻¹). In some embodiments, the kinetic rate constant Kₒₙ describing the association of the query probe with the query region of the target protein to form a complex and/or the kinetic rate constant K_{off} describing the dissociation of the complex is/are great than 0.001 s⁻¹, e.g., great than 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, or 8 s⁻¹.

In certain embodiments, it is possible to automate the delivery of probes to the substrate. High throughput robotic armature in laboratories is routine.

### Samples

A sample can be any composition containing a biomolecule target. Preferably, the biomolecule target is a protein or a plurality of proteins. These proteins include, but are not limited to, polypeptides, peptides, glycoprotein, lipoproteins, microbial polypeptides (e.g., viral, bacterial, or protozoan polypeptides), antibodies, enzymes, disease markers (such as polypeptide cancer antigens), cell surface receptors, hormone receptors, cytokines, chemokines, tissue specific antigens, or fragments of any of the foregoing.

The samples include cell lysates, tissues, tumors, enzymes, biopsy samples, yeasts, fungus, bacteria, plant cells, mammalian cells, circulating tumor cells, biological warfare or terror agents and the like. The content of the sample can be known, characterized and identified, or unknown, uncharacterized and unidentified. In many cases, a sample contains or is suspected of containing one or more enzymatic activities. In other aspects, the sample can be soil remediation sample. A sample can be derived from an organism or can be a man-made. A sample can be, e.g., one containing one or more enzymes in a known quantity or with a known activity. The sample is not a template nucleic acid when the probe is a labeled nucleotide for use in nucleic acid sequencing.

In certain aspects, a biological sample includes tissue sections of normal (colon, lung, etc.) tissue, which can be compared to tissue sections of cancerous (colon, lung, etc.) tissue. Other normal tissues include breast tissue, prostate tissue, kidney tissue, skin, lymph nodes and the like, and can be compared to cancerous tissues of the same tissue type.

### Protein Immobilization

In certain aspects, the substrate or solid support includes any solid or semi-solid material in which a target binding agent such as a plurality of proteins can be attached or incorporated (e.g., physical entrapment, adsorption, and the like) or which can be functionalized to include (e.g., to associate with) a target. Suitable materials include, but are not limited to, a natural or synthetic polymer, resin, metal, or silicate. The proteins can be immobilized in a random fashion or in ordered arrays.

In certain preferred aspects, the present invention provides for a variety of different surfaces (e.g. glass, quartz, plastic) or trapping configurations for immobilizing target molecules for single molecule detection in order to observe probe/target binding kinetics. Suitable chemistries can be used to generate a nonstick surface capable of attaching the target molecules for single molecule detection. The invention provides for detectable probes or detectable targets, such that detection occurs at the single molecule level in order to maximize the determination of kinetic information, in order to acquire “on” and “off” rate constants. Whereas the preferred embodiment immobilizes target molecules such as proteins on a surface, the methods also provide target molecules in solution. The affinity between binding partners can be externally modulated to optimize the affinity information. External modulation can be accomplished by, electric fields, magnetic fields, energy fields, pressure fields, a wash step, convective flow, temperature changes, pH changes, ionic composition or strength changes, and the like.

In one embodiment, the present invention provides a durable surface for target protein immobilization, capable of withstanding multiple wash cycles. In this aspect, the bare surface interacts negligibly with a fluorescent probe, which in turn minimizes background fluorescence and thus “false positive” signals. In certain aspects, a blocking buffer can be used, which is preferably devoid of protein contaminants. In certain preferred aspects, the surface is a thin film (e.g. <30 nm), which keeps proteins nearer the surface for maximal optical excitation; and minimally porous films presenting a flat, compact surface in order to prevent small probes diffusing into the film. In certain aspects, the substrate surface has low binding capacity, which allows for better single-molecule resolution. Certain surface modification is described in US Patent Pub. No. 2009/0175765 to Harris et al. and incorporated herein by reference.

In certain other instances, it is possible to eliminate false positives by subtracting time spectra of the background. This background subtracting is effective in eliminated false positive and increasing the S/N. This procedure also is beneficial to the limit of detection.

In some embodiments, suitable solid-phase support materials include an agarose; a cellulose; a dextran; a polyacrylamide; a polystyrene; a polyethylene glycol; a resin; a silicate; divinylbenzene; methacrylate; polymethacrylate; glass; ceramics; paper; metals; polyacryloylmorpholide; polyamide; poly(tetrafluoroethylene); polyethylene; polypropylene; poly(4-methylbutene); poly(ethylene terephthalate); rayon; nylon; poly(vinyl butyrate); polyvinylidene difluoride (PVDF); silicones; polyformaldehyde; cellulose acetate; nitrocellulose, or a combination thereof. Preferably, the material or combination of materials in the solid support do not interfere with the binding between the probe and the target molecule.

In one embodiment, immobilized carboxylate groups on an amine-reactive surface can be used to covalently link proteins (e.g., with amide bonds) to the substrate via an amine-coupling reaction. Other exemplary reactive linking groups, e.g., hydrazines, hydroxylamines, thiols, carboxylic acids, epoxides, trialkoxysilanes, dialkoxysilanes, and chlorosilanes may be attached to the substrate, such that proteins can form chemical bonds with those linking groups to immobilize them on the substrate.

In certain aspects, the protein is immobilized at one or more lysine amines. In general, protein structures typically contain surface-exposed lysine residues, which are thus available for immobilization chemistry. Proteins not containing lysine are still available to be immobilized via amine chemistry if their amino-terminus is surface-exposed. Alternative immobilization chemistries, such as utilizing the acid side chains of aspartate and glutamate, can also be used.

Various concentrations of proteins can be detected and measured by the methods described herein. Proteins present at concentrations less than, e.g., 100 milligrams/milliliter (mg/ml), 10 mg/ml, 1 mg/ml, 100 micrograms/milliliter (μg/ml), 10 μg/ml, 1 μg/ml, 100 nanograms/milliliter (ng/ml), 10 ng/ml, 1 ng/ml, 100 picograms/milliliter (pg/ml), 10 pg/ml, 1 pg/ml, 100 femtograms/milliliter (fg/ml), 10 fg/ml, or 1 fg/ml are detected in the biological sample, and the concentration can be measured. In fact, in a preferred aspect, a single protein molecule is detected and characterized using the methods of the present invention.

In certain aspects, the protein can be immobilized on a surface using methods such as a hydrophilic self-assembled monolayer approach, a hydrophilic polymer brush approach, a zwiterionic polymer brush approach and a nitrile coating approach. In one aspect, the plurality of proteins are randomly immobilized at a surface density of about 2 proteins to about 1.0×10⁶ proteins per 100 μm×100 μm. In another aspect, the plurality of proteins are randomly immobilized at a surface density of about 2.0×10² proteins to about 8.0×10⁵ proteins per 100 μm×100 μm. In still another aspect, the plurality of proteins are randomly immobilized at a surface density of about 2.0×10³ proteins to about 6.0×10⁴ proteins per 100 μm×100 μm. In still yet another aspect, the plurality of proteins are randomly immobilized at a surface density of about 2.0×10³ proteins to about 1.0×10⁴ proteins per 100 μm×100 μm.

In one embodiment protein molecules are immobilization using a hydrophilic self-assembled monolayer. Proteins are attached to a substrate prior to, or simultaneously with, surface passivation. In this aspect, one suitable linker is commercially available from SoluLinK. Target proteins can be conjugated to a heterobifunctional PEG linker (e.g., PEG-4) conferring a benzaldehyde functionality. The substrate such as glass is activated with for example, hydrazone functional groups. Thereafter, proteins are attached to the surface at low occupancy rates (<0.8 area %) through highly specific, efficient reactions between protein benzaldehyde and surface hydrazone functionalities. Protein occupancy is controlled by protein solution concentration and reaction time and unoccupied surface regions are thereafter passivated with monofunctional (benzaldehyde) PEG chains via the same coupling chemistry used in protein immobilization.

In another embodiment, a surface is prepared that includes a hydrophilic base layer supporting target proteins. In certain aspects, an effective surface film is the polymer “brush,” synthesized directly on the surface from monomers. A suitable chemistry includes “Si-ATRP” (surface initiated atom transfer polymerization), yielding surface-attached polymers of narrow size distribution from aqueous alcohol solutions. Methyl methacrylate derivatives forming polyacrylic acid brushes can be used. A large selection of methacrylate monomers are available commercially (e.g. Sigma-Aldrich) and are suitable. Alternative monomers include, for example, designer peptide surfaces designed for low protein adsorption (Chelmowski, R. et al., J Am Chem Soc, 130(45), 14952-3 (2008)). Published Si-ATRP protocols (Yao, Y. et al., Colloids and Surfaces B: Biointerfaces, 66, 233-239 (2008); Jones, D. M., Huck, W. T. S., Advanced Materials, 13(16), 1256-1259 (2001); Tugulu, S. et al., Biomacromolecules, 6(3), 1602-7 (2005); Edmondson, S. et al., Chem Soc Rev, 33(1), 14-22 (2004); Ma, H. et al., Langmuir, 22(8), 3751-6 (2006); Vaisocherova, H. et al., Anal Chem, 80(20), 7894-901 (2008)) are followed utilizing PEG methacrylate monomers while targeting film thicknesses in the range 20-50 nm. In certain aspects, the hydrophilic brush layer is derivatized with an amine for protein coupling following a published procedure (Yao, Y. et al., Colloids and Surfaces B: Biointerfaces, 66, 233-239 (2008)), optionally followed by a final passivation.

In another embodiment, protein molecules are immobilization using a zwitterionic polymer brush suitable for use in the present invention. In certain aspects, a useful zwitterionic polymer brush has low non-specific protein adsorption of serum proteins (see, Vaisocherova and coworkers (Vaisocherova, H. et al., Anal Chem, 80(20), 7894-901 (2008)). As reported therein, the brush thickness was reproducibly thin at 15-20 nm, which locates interacting probes in the peak energy zone of the TIR optical field. In certain approaches, methods are used that directly link lysines of target proteins to surface carboxylates (Vaisocherova, H. et al., Anal Chem, 80(20), 7894-901 (2008)).

The surface may also be coated with streptavidin at a known concentration, followed by attachment of biotin labeled antibody or a target protein. Streptavidin-biotin interaction is a very high affinity interaction and may be used to immobilize the target proteins. Another antibody to the target protein then may be used to interrogate the target protein antibody interactions.

Proteins immobilized on surfaces remain attached during the analysis. While the preferred embodiment uses chemistries directed toward covalent attachment, adsorbed (non-covalent) proteins are also acceptable. To assess immobilization, surface-attached streptavidin is tagged by exposure to biotin-tagged probes “a0” or “x0”. The protein surface density is adjusted to ensure that at least 90% of probes are bound to protein (i.e. at least 10-fold higher than the background binding to bare surface). After rinsing to remove unbound probes, the surface is imaged in 3 min intervals for several hours in order to estimate the half-life of attached probes. In a complementary method, one immobilizes streptavidin covalently pre-labeled with dye; this eliminates concerns in the alternative method about biotin dissociating from immobilized protein. The effect of dye photobleaching is accounted for statistically by determining the photobleaching rate as before.

The methods of the invention described herein provide proteins (or probes) immobilized on a substrate. The probe can include any substance capable of binding or interacting with a protein. Proteins can bind covalently, non-covalently or not at all to the probe. The probe can be a tumor probe (e.g., PSA) that specifically binds to a protein (e.g., anti-PSA protein). Other tumor-associated proteins that can be immobilized on the surface include, e.g., tyrosinase, MUC1, p53, CEA, pmel/gp100, ErbB-2, MAGE-Al, NY-ESO-1, and TRP-2.

Various proteins, modifications and amino acids are detectable using the present invention. These include, for example, phosphorylation modifications, glycosylation, ubiquitinization, methylation, N-acetylation, lipidation, proteolytic processing, a GPI anchor, a disulfide linkage, a pyroglutamic acid, a nitrotyrosine an acylated amino acid, a hydroxyproline or a sulfated amino acid. A phosphorylated amino acid can be, for example, a phosphoserine, a phosphotyrosine, or a phosphothreonine. These can be detected using either high or low specificity probes of high or low affinity.

Other modification and protein “epitopes” are well known to those of skill in the art, or can be identified using well known methods. Advances in the design of epitope-discovery systems have significantly accelerated the epitope discovery process, giving results quickly. Advanced systems, such as Prolmmune's (www.proimmune.com) REVEAL™ and ProVE™, produce results faster than could be expected with traditional methods. In certain instances, the probes bind protein structure and conformation. For example, certain probes identify and characterize β-sheets, α-helixes and other conformations that are characteristic of proteins. Using the methods herein, it is possible to perform “epitope” mapping of proteins.

### Detection

Detection of binding of a panel of probes to a target protein or polypeptide comprises detection of a label attached directly or indirectly to at least one probe. In certain preferred instances, binding (or absence of binding) between a probe and a protein or a polypeptide to be identified can be detected using single molecule detection methods.

The preferred embodiment utilizes single-molecule imaging methods that provide effective resolution 10-fold, to 20-30 nm (Betzig, E. et al., Science, 313(5793), 1642-5 (2006); Folling, J. et al., Nat Methods, 5(11), 943-5 (2008); Hess, S. T., Girirajan, T. P., and Mason, M. D., Biophys J, 91(11), 4258-72 (2006); Huang, B. et al., Science, 319(5864), 810-3 (2008); Lord, S. J. et al., J Am Chem Soc, 130(29), 9204-5 (2008)). Super-resolution was the TECHNOLOGY OF THE YEAR for 2008 (Hell, S. W., Nat Methods, 6(1), 24-32 (2009); Lippincott-Schwartz, J. and Manley, S., Nat Methods, 6(1), 21-3 (2009)). Other single molecule detection methods are reviewed in Fuller et al., Nature Biotechnology volume 27 number 11 Nov. 2009. These methods rely on various means to randomly switch on fluorescence in a sparse subset of fluorophores populating densely-packed fields. Thus, only a few well-spaced individual molecules are detected in any one image. Subpixel locations are calculated by fitting each imaged diffraction pattern to the theoretical point spread function. Accuracy is typically 20-30 nm, set by the Heisenberg limit Δ/¢m where Δ is the width of the diffraction maximum, and m is the number of detected photons (Folling, J. et al., Nat Methods, 5(11), 943-5 (2008)). Different fluorophore subsets are detected in subsequent images, a composition of which reveals the densely-packed field with super-resolution thus the size of a typical protein (10 nm). The invention provides a natural compatibility with super-resolution imaging because only a subset of proteins are detected in any given image. Fluorophore switching is not necessary. The field is naturally parsed both spatially and temporally by probes binding only a subset of the proteins and by probes binding neighboring proteins at different moments in time with on-rates dependent on probe concentration. Diffraction patterns, obtained by summing repeated binding events in movies, are detected with high photon counts m, which supports locating proteins with high accuracy (A/Cm). In a preferred embodiment of about 30 nm spatial resolution, one million proteins are immobilized with 80% resolution, rendering 800,000 proteins for analysis.

The maximum packing density, defined herein where 80% of proteins are resolvable, occurs with just 7900 total proteins yielding 6300 optically resolved proteins in the field of view.

Total internal reflectance fluorescence (TIRF) microscopy is a preferred detection device of the present invention. TIRF provides an optical effect that can be used to observe fluorescent events occurring at the interface between two optical media of different refractive indices. Excitation laser induced light incident upon such a boundary, travelling at an angle greater than the critical angle, undergoes total reflection. The total internal reflected light extends into the sample on the substrate beyond the interface, extending only a few hundred nanometers into the second medium of lower refractive index (e.g., the z direction). This evanescent field allows for fluorescence excitation. The excitation volume of a TIRF evanescence field extends about 100 nm into the sample. Photons that are created within that excitation volume from the unique labels of the probes, are detected. TIRF microscopy (TIRFM) is used for capturing a high resolution, high signal to noise (S/N) series of binding events as claimed herein. The single molecule detection has spatial resolution at about 1 nm to about 100 nm, preferably about 5 nm to about 50 nm and more preferably at about 10 nm to about 40 nm.

Single molecule detection (SMD) allows for a high degree of multiplexing (e.g. 1 million per 100×100 micron field of view using super-resolution computational imaging). Further, SMD enables counting individual binding pairs and correlating the counts to biological relevance as well as kinetic “on” and “off” rates measured on many individual molecules simultaneously, allowing the assessment of N different types of targets with <N unique probes. Moreover, SMD allows kinetics to be measured without the limitations of equilibrium averages characteristic of ensemble based assays. A variety of detection modalities can be used, including fluorescence, FRET, multi-photon, polarization, plasmonic effects, AFM, force spectroscopy, fluorescence lifetime, light scattering, Raman scattering, and the like.

In an embodiment, the methods provide minimization of background binding of probes to the bare working surface by utilizing protein immobilization surface chemistries that provide a non-stick film supporting the covalent attachment of target proteins. The preferred embodiment of the invention also provides reproducibility in measurements of transient interactions. The background flux of transient binding events are preferably limited to less than 0.05 um⁻² s⁻¹. The background flux of static binding events are preferably less than 0.0005 um⁻² s⁻¹. The protein immobilized half-life is typically greater than 5 hours. The target proteins are reproducibly distinguished with at least 99% confidence by transient binding.

In certain instances, transient binding interactions are characterized by mathematical transformations of the rate constants, such as autocorrelation histograms, without directly computing the rate constants themselves. Statistical matrices such as Possion statistics, hidden Markov modeling or edge detection algorithm may be sued. Also, useful are statistical matrices such as mean, median, peak and standard deviations of both residence time and the number of transient events measured for the repeated binding of the probes with the protein molecules at plurality of locations.

### Applications and Uses of the Methods

The invention can also detect modifications such as glycosylation, ubiquitinization, methylation, N-acetylation, lipidation, and proteolytic processing, using either high or low specificity probes of high or low affinity. In addition to protein analysis, the invention detects other binding pairs, i.e. lipids, nucleic acids, inorganic molecules, drugs, environmental molecules (e.g., explosives, toxins).

Biomarkers in disease. The invention allows case and control samples to be compared to identify immobilized proteins correlated with the disease state. These biomarker proteins are then subsequently purified in order to determine their biological identity.

Biomarkers in the environment. Environmental samples which are collected over time are assayed by the invention to monitor variation in marine, terrestrial, and soil environments. It is possible to correlate proteins with changing ecosystem compositions.

In certain embodiments, the methods described herein are used to evaluate the efficacy of treatment of a disease of a subject. Such an evaluation includes, e.g., obtaining at least one biological sample from the subject typically before treatment begins, as well as obtaining at least one biological sample from the subject any time after commencement of the treatment or therapy. The pre- and post-treatment samples are then evaluated using the methods to characterize at least one protein or probe that is indicative of the disease. The efficacy or success of treatment is evaluated by comparing the amount, or change in protein or probe in each sample. For example, a decrease in the amount of the protein in the sample obtained after treatment commenced is an indication that the treatment or therapy of the disease is efficacious. The presence of proteins (e.g., antibodies) produced in a subject during treatment of a disease is determined using the methods described herein, e.g., to determine the onset or extent of resistance to treatment.

### EXAMPLES

### Example 1

Biological samples: Spike 1, 5, 10, 50, 100, 200, 500, 1000, 5000, or 10,000 fg of IL-6 in 1 ml of IL-6 deprived human serum to generate a group of samples with different concentrations of IL-6. Mix 1 nM of biotinylated high affinity anti-IL-6 antibody (clone C01, Bio-Rad SAC) with 10 μL of each IL-6 spiked serum sample. Incubate for 30 min at room temp to allow the antibodies to capture the IL-6 molecules. The antigen-antibody complex is then presented to a prism surface functionalized with streptavidin. The IL-6 molecules are immobilized to prism surface in this process. A Cy5 or ATTO680-conjugated high off-rate IL-6 antibody (Clone 295.9, off-rate 0.2, Bio-Rad) is added to the prism surface at a non-saturating concentration (5 μM). A short period incubation time (10 min) is given to allow the interaction between detection antibodies and antigens to reach equilibrium. The prism surface is then observed under a high spatial resolution prism-type TIRF microscope which allows fluorescent detection of single molecules within about 200 nm to the prism surface. A series of images of the same view field (up to 100 μm×100 μm) are captured at a rate of 2 Hz using an EMCCD camera for 10 min. Computational analysis of the data reveals the pattern of the fluorescent signal profile over the period of 10 min for each singe molecule at its specific location. This profile is a reflection of the kinetic fingerprinting of the antibody to this specific molecule. The fluorescent profile of a specific binding of the antibody to its antigen would be significantly different from any nonspecific interactions (e.g. interaction frequency, on-time, etc.). By examining the profiles of all single molecules detected in the view field, the real/specific signal is isolated from the noises and by counting digitally how many antigen molecules are detected on the TIRF prism surface.

FIG. 1(A) shows a thermodynamic profile of the IL-6 antibody interacting with a spot through non-specific binding. The total number of on- and off-events of the antibody at this spot is 6.

FIG. 1(B) shows a thermodynamic profile of the IL-6 antibody interacting with an IL-6 molecule on the surface. The total number of on- and off-events of the antibody at this spot is 24, significantly higher than the number for a non-specific spot.

In FIG. 1(C), the IL-6 antibody interacted with a total of 2900 individual locations on the assay surface and generated 2900 individual thermodynamic profiles. Out of these, 2500 profiles (non-specific binding population) showed low probability of the IL-6 antibody interacting with them. There were a total of 2-10 on- and off-events observed for these spots. For the rest of the 400 spots (specific binding population), the IL-6 antibody actively seeked interaction with them which is demonstrated by a total of 20-24 on- and off-events. This results show that 400 target molecules are detected on the assay surface.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for characterizing the binding characteristic of at least one protein molecule to a detectably labeled probe, the method comprising:
a. contacting the protein molecules with a detectably labeled probe which exhibits fast-off rate binding characteristics with respect to the protein, to generate a transient binding interaction between the protein molecule and the probe;
b. detecting the transient interactions by single molecule detection of the labeled probe at plurality of locations, wherein the transient interactions have an observed residence time of about 10 minutes to about 1 nanosecond; and
c. correlating the interaction frequencies of multiple transient interactions at the plurality of locations to determine the binding characteristic of the protein molecule to the probe.

2. The method of claim 1, wherein the protein is immobilized on a surface.

3. The method of claim 2, wherein the immobilized surface is glass, quartz or plastic.

4. The method of claim 3, wherein the protein is immobilized on the surface using a hydrophilic self-assembled monolayer, a hydrophilic polymer brush, a zwitterionic polymer brush or a nitrile coating.

5. The method of claim 3, wherein the surface is coated with streptavidin and the protein is immobilized to the surface using biotinylated protein.

6. The method of claim 2, wherein the protein is immobilized at a surface density of about 2 molecules to about 1×10⁶ molecules per 100 μm².

7. The method of claim 5, wherein the protein is immobilized at a surface density of about 2×10² molecules to about 8×10⁵ molecules per 100 μm².

8. The method of claim 6, wherein the protein is immobilized at a surface density of about 2×10³ molecules to about 6×10⁴ molecules per 100 μm².

9. The method of claim 1, wherein correlating the interaction frequencies of multiple transient interactions at the plurality of locations allows separation of protein-specific signal from nonspecific noise.

10. The method of claim 9, wherein ratio of protein-specific signal interactions to nonspecific noise interactions is more than or about 2.

11. The method of claim 10, wherein ratio of protein-specific signal interactions to nonspecific noise interactions is more than or about 3.

12. The method of claim 11, wherein ratio of protein-specific signal interactions to nonspecific noise interactions is more than or about 4.

13. The method of claim 1, wherein correlating the interaction frequencies of multiple transient interactions at the plurality of locations measures total number of on- and off-events at plurality of locations.

14. The method of claim 13, wherein more than 1000 transient interactions are recorded per viewing area.

15. The method of claim 14, wherein more than 2000 transient interactions are recorded per viewing area.

16. The method of claim 15, wherein more than 4000 transient interactions are recorded per viewing area.

17. The method of claim 1, wherein the binding characteristic comprises a statistical metric calculated from the multiple transient interactions between the probe and the protein molecules at the plurality of locations.

18. The method of claim 17, wherein the statistical metric is calculated from Poisson statistics, hidden Markov modeling or an edge detection algorithm.

19. The method of claim 17, wherein the statistical metric comprises one or more of:
a. a mean of a distribution of residence times measured for the repeated binding of the probes with the protein molecules at plurality of locations;
b. a median of a distribution of residence times measured for the repeated binding of the probes with the protein molecules at plurality of locations;
c. a standard deviation of a distribution of residence times measured for the repeated binding of the probes with the protein molecules at plurality of locations;
d. a peak of a distribution of residence times measured for the repeated binding of the probes with the protein molecules at plurality of locations;
e. a shape of a distribution of residence times measured for the repeated binding of the probes with the protein molecules at plurality of locations;
f. a mean of a distribution of number of transient events measured for the repeated binding of the probes with the protein molecules at plurality of locations;
g. a median of a distribution of number of transient events measured for the repeated binding of the probes with the protein molecules at plurality of locations;
h. a standard deviation of a distribution of number of transient events measured for the repeated binding of the probes with the protein molecules at plurality of locations;
i. a peak of a distribution of number of transient events measured for the repeated binding of the probes with the protein molecules at plurality of locations; or
j. a shape of a distribution of number of transient events measured for the repeated binding of the probes with the protein molecules at plurality of locations.
